(19) United States
(12) Patent Application Publication
(10) Pub. No.: US 2012/0039947 A1
(43) Pub. Date: Feb. 16, 2012

(54) METHODS AND COMPOSITIONS FOR TREATING PSORIASIS

(75) Inventor: Joel E. Bernstein, Deerfield, IL (US)

(73) Assignee: ELORAC, LTD, Vernon Hills, IL (US)

(21) Appl. No.: 12/854,788

(22) Filed: Aug. 11, 2010

(51) Int. Cl. A61K 9/00; A61P 17/06; A61K 31/122

(52) U.S. Cl. 424/400; 514/680

(57) ABSTRACT

Methods and compositions including anthralin for treating psoriasis of the skin and scalp are described.

### BACKGROUND

Psoriasis is a chronic papulosquamous cutaneous disorder that has been the object of a variety of oral and topical treatments. Included among these treatments is topically applied anthralin, a distant relative of the generational old treatment, coal tar. While anthralin formulated into creams, ointments and lotions has been demonstrated to be an effective modality for the control of plaque-type psoriasis, its use has been severely limited by several unpleasant properties of anthralin:

(a) a high incidence of moderate to severe irritation of the skin, especially the unaffected skin surrounding psoriatic plaques;

(b) a propensity to stain skin yellow, orange or black, as well as similarly staining fabrics and bathroom fixtures; and

(c) commercial formulations turn from yellow-orange to dark orange or black in the tube over the course of use.

Psoriasis involves the scalp in nearly all cases. The involvement of the scalp is characterized by individual or diffuse red plaques covered with a slight to very heavy silvery scale. These scales are shed and the result is a very unsightly appearance of the hair and scalp. Shampoos with salicylic acid, coal tar recently corticosteroids, have been utilized in attempts to control scalp psoriasis with modest success.

Anthralin incorporated into creams, ointments and suspensions has been applied to psoriatic lesions of the scalp, allowed to remain on the scalp for a prolonged time and then removed by shampooing with an unmedicated shampoo. Such a regimen usually leads to unacceptable irritation of the scalp, as well as a particularly inconvenient regimen to follow. Prior attempts to prepare stable formulations of anthralin-containing shampoos have been heretofore unsuccessful primarily due to the surfactants in a shampoo rapidly degrading the anthralin.

In order to surmount these problems, I have prepared a variety of liquid anthralin formulations including creams, gels, ointments, lotions and suspensions with anthralin particles encapsulated within a crystalline coating of glyceryl monolaurate and glyceryl monomyristate and with blue dyes incorporated into the compositions. Some of the suspensions include surface active and sudsing agents so that the resulting composition can be used as a shampoo.

I have discovered that by encapsulating the anthralin particles in spheres composed of glyceryl monolaurate and glyceryl monomyristate and by incorporating these spheres in a surrounding matrix containing a blue dye, the resulting formulation produces less irritation than traditional anthralin formulations as well as virtually no orange to rust staining of the skin.

### SUMMARY OF THE DISCLOSURE

Methods and compositions including anthralin for treating psoriasis of the skin and scalp are described.

A composition for treatment of psoriasis includes anthralin encapsulated by one or more monoglycerides and dispersed in water in oil, or oil in water emulsions containing a blue dye. The composition has comparable efficacy to other anthralin compositions, but with significantly less adverse effects. Adverse effects of ahthralin include skin irritation, staining of skin, fabric and fixtures, and deterioration of the color of the composition.

Suitable encapsulating monoglycerides include glycerol monolaurate, glyceryl monomyristate and related monoglycerides.

A vehicle to deliver the composition may be a lotion, a solution, a suspension, a cream, an ointment or a gel.

Anthralin is present in an amount of about 0.01% to about 5.0% by weight.

The blue dye is an FD&C blue dye, for example FD&C Blue #1 or FD&C Blue #2.

The blue dye is present in a concentration of about 0.01% by weight to about 5.0% by weight.

When the composition is a shampoo, surfactants may be incorporated into the shampoo. Surfactants include sucrose stearate, glycereth-2 cocoate, related monoglycerides, and combinations thereof.

The shampoo includes about 0.01% to about 5.0% by weight of an FD&C blue dye.

A method for treating psoriasis of the skin includes the steps of:

(a) applying an anthralin composition for a period of from 10-30 minutes once or twice daily; and

(b) removing the composition by washing with soap and water immediately after each 10-30 minute application period.

A method for treating psoriasis of the scalp includes the steps of:

(a) lathering the scalp and hair with shampoo anthralin; and

(b) contracting the composition in a lather form to the scalp for from 1 to 10 minutes; and

(c) rinsing the scalp thoroughly with water.

Application of the shampoo may be repeated from 2-3 times a week or daily.

Methods and compositions for treating psoriasis are described that reduce the irritation of the skin produced by anthralin and reduce the incidence of orange to rust staining which may accompany anthralin therapy.

Methods and compositions are described for preparing stable shampoo formulations containing anthralin as the active ingredient, along with a novel treatment regimen for use of such shampoos.

These and other objects of the present invention may be more readily understood when considered with the following detailed description and examples.

### DETAILED DESCRIPTION OF THE DISCLOSURE

It has been discovered surprisingly that incorporating anthralin into monoglyceride capsules and dispersing these capsules into a blue cream, suspension, ointment, gel or lotion base, and then applying such formulations for the treatment of psoriasis, reduces the irritation of the skin often caused by anthralin, reduces or even eliminate the propensity of anthralin products to stain skin as well as fabrics and bathroom fixtures, and prevents commercial formulations from turning an unpleasant dark orange or black in the tube over time.

Creams, ointments, lotions, gels and suspensions are prepared employing 0.05% by weight to 5% by weight concentrations of anthralin in aqueous vehicles containing about 2% to 12% of glyceryl monolaurate and 5% to 36% by weight of glyceryl monomyristate, along with 0.01% to 5% of FDA approved blue dyes. The anthralin particles are encapsulated by spheres composed of glyceryl monolaurate and glyceryl monomyristate by heating the anthralin with these monoglycerides to a temperature of from 50° to 80° Centigrade for from 5 to 30 minutes, and then cooling slowly until the mixture reaches ambient temperature. The resulting mixture is then incorporated into aqueous liquid vehicles containing FDA approved blue dyes rendering the resulting formulations green.

Anthralin incorporated into monoglyceride capsules is useful to formulate stable shampoos. The monoglyceride coating protects anthralin from degradation by the shampoo surfactants. Washing the scalp with such shampoos is surprisingly effective in removing psoriatic scales and reducing erythema of psoriatic scalp plaques even though the contact time for the anthralin with the scalp is unusually short (from about 1-10 minutes).

Monoglyceride-encapsulated anthralin is incorporated into shampoo vehicles Such shampoos are used to wash the hair resulting in only very brief contact of the anthralin with the scalp. Such brief shampooing (for 1-10 minutes) once daily or even in severe cases every other day provides exceptionally good control of scalp psoriasis. The concentration of anthralin incorporated into the shampoo is from about 0.01 to about 5.0% by weight and preferably from about 0.25% to about 1.5% by weight.

### EXAMPLES

Examples are provided for illustrative purposes and are not intended to limit the scope of the disclosure.

### Example 1

A mixture of 7% glyceryl monolaurate and 21% glyceryl monomyristate and 1.2% anthralin were mixed under a nitrogen blanket. The resultant mixture was heated to 65° C. for 60 minutes and allowed to cool. A buffer solution was then added to this mixture and this formulation was stirred while heated to 50-80° C. for an additional 10-30 minutes and again allowed to cool. The resulting cream formulation was applied to psoriatic plaques of patients suffering from psoriasis for from 10 to 30 minutes once or twice daily and then washed off with soap and water. The skin of patients treated in such fashion demonstrated only rare instances of irritation and no orange to rust staining.

### Example 2

A mixture of 10% glyceryl monolaurate and 30% glyceryl monomyristate and 5% anthralin was mixed with a buffer solution, heated to a temperature of 80° C. for 30 minutes and allowed to cool to room temperature. The resulting ointment formulation was applied once or twice daily to psoriatic lesions and removed by washing with soap and water after about 10 minutes. After 4 weeks of such therapy, the psoriatic lesions resolved without residual irritation or staining of the skin.

### Example 3

A mixture of 0.01% anthralin, 9% glyceryl monolaurate and 25% glyceryl monomyristate was heated to 65° C. for 30 minutes and then cooled. A buffer solution containing the surfactants sucrose stearate and glycereth-2 cocoate along with FD&C Blue #1 were then added. The resulting shampoo formulation was then utilized by patients with psoriasis of the scalp from once daily to 2-3 times per week. The shampoo was lathered into the scalp for about 1-10 minutes and then rinsed off with water. Such a regimen is highly effective for psoriasis of the scalp and the shampoo produces only very rare irritation without orange to rust staining of the scalp.

1. A composition for treatment of psoriasis, the method comprising anthralin encapsulated by one or more monoglycerides and dispersed in water in oil, or oil in water emulsions, and containing a blue dye, wherein the composition has comparable efficacy to other anthralin compositions, but with significantly less adverse effects.

2. The composition of claim 1, wherein adverse effects are selected from the group consisting of skin irritation, staining of skin, fabric and fixtures, and deterioration of the color of the composition.

3. The composition of claim 1, wherein the encapsulating monoglycerides are selected from the group consisting of glycerol monolaurate, glyceryl monomyristate, related monoglycerides and combinations thereof.

4. The composition of claim 1 wherein said composition is suitable for application to the skin.

5. The composition of claim 1 further comprising a vehicle.

6. The composition of claim 5, wherein the vehicle is selected from a group consisting of a lotion, a solution, a suspension, a cream, an ointment and a gel.

7. The composition of claim 1 wherein anthralin is present in an amount of about 0.01% to about 5.0% by weight.

8. The composition of claim 1 wherein the blue dye is an FD&C blue dye.

9. The composition of claim 8 wherein the FD&C Blue dye is selected from the group consisting of FD&C Blue #1 and FD&C Blue #2.

10. The composition of claim 1 wherein the blue dye is present in a concentration of about 0.01% by weight to about 5.0% by weight.

11. The composition of claim 1 is a shampoo.

12. The composition of claim 11 wherein surfactants are incorporated into the shampoo.

13. The composition of claim 12 wherein the surfactants are selected from the group consisting of sucrose stearate, glycereth-2 cocoate, related monoglycerides, and combinations thereof.

14. The composition of claim 11, wherein the shampoo comprises about 0.01% to about 5.0% by weight of an FD&C blue dye.

15. A method for treating psoriasis of the skin, the method comprising:
(a) applying a composition of claim 1 for a period of from 10-30 minutes once or twice daily; and
(b) removing the composition by washing with soap and water after each application.

16. A method for treating psoriasis of the scalp, the method comprising:
(a) lathering the scalp and hair with a shampoo composition as provided in claim 11;
(b) rinsing the scalp thoroughly with water; and
(b) contacting the composition to the scalp from about 1 to 10 minutes.

17. The method of claim 16, repeated from 2-3 times a week.

18. The method of claim 16 repeated daily.
